(19) United States
(12) Patent Application Publication
(10) Pub. No.: US 2013/0092165 A1
(43) Pub. Date: Apr. 18, 2013

(54) Nasal Ventilation Cannula System and Methods

(71) Applicant: Anthony D. Wondka, Thousand Oaks, CA (US)

(72) Inventor: Anthony D. Wondka, Thousand Oaks, CA (US)

(73) Assignee: Anthony David Wondka, Thousand Oaks, CA (US)

(21) Appl. No.: 13/628,038

(22) Filed: Sep. 26, 2012

Related U.S. Application Data
(60) Provisional application No. 61/539,228, filed on Sep. 26, 2011.

(51) Int. Cl. A61M 15/08; A61M 16/00

(52) U.S. Cl. CPC A61M 15/08; A61M 16/0057
USPC 128/204.25

(57) ABSTRACT

A nasal cannula ventilation system is described for treating lung disease or for exercise conditioning, incorporating a Venturi system. The ventilation cannula comprises unique positioning features to positively locate a gas delivery nozzle in an optimal location to optimize Venturi performance, patient comfort and fitment to the patient. The cannula is low profile, making it as realistic to wear and use as a standard oxygen cannula, and is simple rending the cost reasonable. The ventilation cannula uses a simple low cost ventilator as a gas delivery control system which is compatible with existing gas sources. The system is used (1) during stationary use to unrest the respiratory muscles to increase tolerance to activity after a treatment session, or (2) to enable activity within a distance from a stationary gas source, (3) during ambulatory use using a portable gas source to enable mobility, and (4) for enhanced fitness conditioning.

### PRIORITY CLAIM

This application claims priority to U.S. Provisional Patent Application No. 61/539,228 filed on Sep. 26, 2011.

### FIELD OF THE INVENTION

The present invention relates to the field of improving ventilation to improve exercise capacity in fitness training, and or to increase exertion tolerance in the treatment of lung disease such as chronic obstructive pulmonary disease (COPD), interstitial lung disease (ILD) and other respiratory disorders. More specifically, this invention involves delivering supplemental oxygen to a person under enough pressure to decrease the work of breathing and or to increase exercise capacity. The invention employees a nasal ventilation cannula with a unique minimally encumbering system that delivers the oxygen with a Venturi effect to improve the efficiency of respiration and ventilation.

### BACKGROUND OF THE INVENTION

Regarding fitness training, increasing exercise capacity using supplemental aides to obtain a competitive advantage is a growing phenomenon. Mechanical non-pharmacological aides to accomplish this are very few, forcing athletes to be tempted with illegal doping. Athletes that use legal techniques are left with inconvenient options, for example living in a high elevation before a competition, or sleeping in a tent with a special atmosphere. A supplemental tool that could be conveniently accessed and used would be a vast improvement over the state of the art.

Regarding lung disease, COPD and ILD are worldwide problems of high prevalence, and cause significant health care costs to society. COPD is a progressive disease in which there is significant mechanical disadvantage of the breathing apparatus. ILD is also a progressive disease in which the lungs become stiff and resistive. In both diseases the work of breathing increases. Patients are able to ventilate themselves at rest, however, with activity, because of the increased ventilatory demand combined with degraded lung mechanics, patients cannot support their ventilatory needs and are forced to immediately suspend their physical activity in order to survive. Current prescribed therapies for COPD and ILD include pharmacological agents (beta-agonist aerosolized bronchodilators and anti-inflammatories), supplemental nasal oxygen therapy, pulmonary rehabilitation, pulmonary hygiene (lavage, percussion therapy), lung volume reduction and lung transplantation. These therapies all have certain disadvantages and limitations with regard to user adherence, effectiveness, risk or availability. Usually, after progressive decline in lung function regardless of the therapeutic pathway chosen, patients become physically incapacitated and sometimes require mechanical ventilation to survive, after which weaning from ventilator dependency is difficult. Mechanical ventilation could be used to provide the needed ventilatory support, however, it is not used either because either (a) the equipment is incompatible with activity, or (b) the patient interface is too obtrusive for a patient to realistically use on an elective basis, or (c) as in the case of CPAP, reimbursement is not widely established.

In the past non-invasive ventilation (NIV) in conjunction with applying CPAP via a nasal mask or face mask at night has been used successfully used to treat COPD. The therapy works by unloading and resting the respiratory muscles, thereby allowing the patient to be more active the following day. Known as nocturnal CPAP, the potential efficacy of this therapy was well reported by the medical community in small non-controlled non-statistically powered studies in the 1990's (Petrof B J; Am Rev Respir Dis. 1991 May; 143; 5 Pt 1:928-35). However in 2006, per their guidelines Medicare asked for controlled clinical evidence for the efficacy of nocturnal CPAP in order to continue reimbursement. Unfortunately the manufacturers distributing this equipment did not have the required clinical data, forcing Medicare to evaluate the merits of the therapy on small, physician-sponsored non-statistically powered studies. As a result, Medicare approved reimbursement for only a very narrow population of COPD patients for which data was available, and henceforth this therapy became unavailable to thousands of patients who could have benefited from it. Controlled clinical studies are now being planned in the hope of resurrecting this therapy.

Other forms of respiratory support using non-sealing or non-cumbersome masks have been described. High flow oxygen therapy (HFOT) has been successfully used as a hospital based therapy, delivering 15 lpm+ of humidified oxygen to the patient using a non-mask nasal delivery cannula. It has been proven to lower the work of breathing (Criner G J, “Ventilatory muscle recruitment in exercise with O2 in obstructed patients with mild hypoxemia.” J Appl Physiol 1987; 63:195-200). Technologically, because the therapy requires at least 15 lpm of flow at pressures of less than 20cmH2O, the nasal cannula needed is relatively large to accommodate that flow rate, making the therapy less desirable than the invention described herein. Because of its high cost HFOT may be limited to the hospital patient being weaned from mechanical ventilation, or in attempt to avoid mechanical ventilation—in these clinical situations the DRG payment system provides adequate funds for providers to employ the therapy. Transtracheal oxygen therapy (TTOT) has been successfully used as a hospital based therapy, also to wean a patient from mechanical ventilation. TTOT has the same limitations as HFOT and because it requires a tracheotomy it is further limited in its use.

Non-invasive open ventilation (N-IOV) is a variant of NIV that is being developed (Genger, U.S. Pat. No. 7,080,645; Matarasso, U.S. Pat. No. 7,562,659; Wondka, US Patent Application No. 20100252037; and US Patent Application No. 20110094518). N-IOV shows promise to treat and improve the exertion tolerance in debilitated COPD and ILD patients. N-IOV works by a Venturi principle, using a non-sealing nasal mask which delivers pressurized oxygen gas through a gas delivery jet nozzle, which creates a Venturi effect and entrains ambient air into the patient's nasal airway. Relatively small amounts of oxygen gas can be used to potentially create a commensurate moderate level of pressure support in the lungs. In non-controlled studies, this therapy has been shown to improve the six minute walk distance of exertion limited patients (“Improved 6MWT distance with a highly portable non-invasive ventilator”, Hilling, Wondka et. al., Am J Respir Crit Care Med 181;2010:A1198).

Eventually there will be biological treatments and potentially even cures for COPD and ILD using biotechnology approaches such as stem cell therapy, genetic therapies, or other techniques. However these interventions are at least 20 years away from being developed, tested and approved. Therefore, until those treatments are available, there is a need for a more user-friendly ventilation system to treat patients with COPD and ILD to restore activity levels and reduce breathing effort, dyspnea and fatigue. Ideally, this new ventilation therapy would provide the needed ventilatory support, but with a non-cumbersome patient interface that does not seal the airway, and in a form factor that permits patient adherence. Ideally, the therapy's technology should be designed such the therapy can be used during physical activity and/or during ambulation; or exercise to enable activities of daily living to allow the patient to continue to contribute to society; and to allow the patient to become healthier overall. This therapy would then lower healthcare costs to society by making this very large group of patients less dependent on expensive sudden medical interventions when their symptoms become exacerbated. The therapy would also ideally meet the needs of the healthcare delivery stakeholders by being low cost to deploy and maintain.

### SUMMARY OF THE INVENTION

Nasal cannulae used for supplemental oxygen delivery are common and accepted among users and the general public, however, a nasal cannula does not enhance ventilation due to its lack of delivery power. In contrast, nasal mask ventilation enhances ventilation by delivering the gas under power, but these systems are undesirable to users because of their obtrusiveness, and they are generally ostracized by the general public. The present invention describes a nasal cannula ventilation system, or NCV, which incorporates a unique Venturi system into a nasal oxygen cannula to deliver gas to a patient under power to enhance ventilation, thereby creating a ventilation therapy device from a platform that has a proven track record of patient adherence and which can be very low cost to deploy. NCV uses a ventilation cannula is exceptionally low profile and un-encumbering, making it practical to be electively used by the patient. The ventilation system will typically comprise a simple ventilator or gas delivery control system that is of a small form factor that can be attached to an oxygen supply, and optionally toted and/or worn by the patient. The goal of the system when used to treat lung disease is to (a) rest the respiratory muscles when using during stationary therapy sessions so that the user has more respiratory reserve and can be more active after the treatment; (b) allow the user to participate in activities of daily living by provide relief of dyspnea and fatigue and contributing to the work of breathing during semi-stationary activities like bathing; (c) provide mechanical respiratory support to enable the patient to engage in non-stationary activity such as ambulation; (d) improve the pulmonary conditioning of the user by providing mechanical respiratory support during exercise sessions. The goal of the system when used for fitness training is to provide more oxygen and greater breathing volumes during maximal exercise so that the systemic muscles can produce additional work beyond their normal peak work levels in order to improve the conditioning of the overall vasculature system and muscular system.

In a first main embodiment of the present invention the nasal ventilation cannula comprises a dual ported distal tip, with one port for sensing breathing, and the other port a jet nozzle for delivering gas at high velocity. The sensing port tip is positioned in a configuration that (1) maximizes sensing fidelity, (2) maximizes comfort to the user, and most importantly (3) positionally indexes the gas delivery jet nozzle in a position that [a] optimizes the Venturi effectiveness and [b] optimizes the comfort of the sensation of therapy to the user.

In a second main embodiment of the invention the nasal ventilation cannula sensing port tips include a configuration that optimizes the Venturi effectiveness of the gas flow profile of the gas exiting the jet nozzle.

In a third main embodiment of the invention, the gas delivery nozzle comprises functional features to create a gas profile that matches the shape of the nostril and intersects with the nostril wall at a desired distance inside the nostril.

In a forth embodiment of the invention, oxygen is delivered through the sensing port for oxygenation, and air is delivered through the jet nozzle for mechanical ventilatory support.

In a fifth embodiment of the invention, a gas delivery system controls the therapy and is attached to a medical gas source, such as oxygen or air.

In a sixth embodiment of the invention the nasal ventilation cannula incorporates special features to optimize the Venturi and reduce shear forces created by the jet and therefore reducing shear-related sound.

In a seventh embodiment, the system is integrated into an existing medical oxygen gas delivery system such as a portable Oxygen cylinder.

### BRIEF DESCRIPTION OF THE FIGURES

FIG. 1 describes the ventilation cannula being worn by a patient user.

FIG. 2 shows an isometric view of the ventilation cannula assembly.

FIG. 3 describes a front view of the distal end of the ventilation cannula of detail A shown in FIG. 1.

FIG. 4 shows a front cross-sectional view of the nose and distal end of the ventilation cannula shown in FIG. 3.

FIG. 5 describes a sectional top view through the user's nose of distal end of the ventilation cannula at line C-C shown in FIG. 3.

FIG. 6 describes an optional configuration of the distal end of the ventilation cannula shown at Detail A of FIG. 1.

FIG. 7 describes a front view of the distal end of the ventilation cannula shown in FIG. 6.

FIG. 8 describes a schematic sectional front view of the user's nose and the distal end of the ventilation cannula during expiratory phase.

FIG. 9 describes a schematic sectional front view of the user's nose and the distal end of the ventilation cannula during pressure support phase, or during inspiratory phase.

FIG. 10 describes a top view of the distal end of a nasal ventilation cannula with medial gas sensing tubes and centered ventilation gas delivery ports.

FIG. 11 shows a front sectional view of the ventilation cannula shown in FIG. 10.

FIG. 12 shows a sectional front view of alternate ventilation cannula to the cannula shown in FIG. 1 in which the gas delivery ports are medial and the sensing ports are centered.

FIG. 13 shows a top view of a ventilation cannula with lateral sensing ports and centered ventilation gas delivery ports.

FIG. 14 shows a front sectional view of the ventilation cannula shown in FIG. 13.

FIG. 15 describes an optional end view of the gas delivery nozzle including a sound diffusing radius at the nozzle tip and sound reducing dimples along the inside edge of the diffuser.

FIG. 16 describes a cross sectional side view of the gas delivery nozzle shown in FIG. 8.

FIG. 17 shows a ventilation cannula with patient circuit with a dual lumen construction.

FIG. 18 describes a cross-sectional view of the ventilation cannula at line E-E shown in FIG. 17, showing a dual lumen construction.

FIG. 19 describes a cross-sectional view of the ventilation cannula at line E-E shown in FIG. 17, showing a quintuple lumen construction.

FIG. 20 describes an alternate cross-sectional view of the ventilation cannula showing a side-by-side tubing construction.

FIG. 21 describes a cross-sectional view of the ventilation cannula at line E-E shown in FIG. 17, showing a non-round dual lumen construction.

FIG. 22 describes an alternate cross-sectional view of the ventilation cannula showing a dual tube lumen construction at the distal tip, wherein the ventilation gas delivery tube is placed inside a pocket in the breath sensing tube. The sensing tube and gas delivery tube can be molded or formed separately, then assembled together.

FIG. 23 shows a ventilation cannula with a patient circuit with a dual tube construction, with a first side assigned to ventilation gas delivery and an opposite side assigned to breath sensing.

FIG. 24 describes a cross-sectional view of the dual tube construction ventilation cannula at line G-G shown in FIG. 23, showing the ventilation gas delivery tube cross-section.

FIG. 25 describes a cross-sectional view of the dual tube construction ventilation cannula at line G-G shown in FIG. 23, showing the breath sensing tube cross-section.

FIG. 26 shows a top view of the left side of the distal end of the ventilation cannula at Detail B shown in FIG. 2, showing a round dual lumen configuration with a lumen terminating with a breath sensing port and a lumen terminating in the gas delivery nozzle.

FIG. 27 shows a front view of the ventilation cannula detail shown in FIG. 26.

FIG. 28 shows a top view of the left side of the distal end of the ventilation cannula at Detail B shown in FIG. 2, showing a non-round dual lumen configuration with a lumen terminating with a breath sensing port and a lumen terminating in the gas delivery nozzle.

FIG. 29 shows a front view of the ventilation cannula detail shown in FIG. 28.

FIG. 30 shows a top view of the left side of the distal end of the ventilation cannula at Detail B shown in FIG. 2, showing an adjoining dual tube configuration with a tube terminating with a breath sensing port and a tube terminating in the gas delivery nozzle.

FIG. 31 shows a front view of the ventilation cannula detail shown in FIG. 30.

FIG. 32 describes a front view of the left side of the distal end of the ventilation cannula at Detail B shown in FIG. 2, showing a shield to shield the nostril inner wall from the jet gas flow.

FIG. 33 describes a front view of the distal end of a ventilation cannula with a Venturi throat coupled to the cannula.

FIG. 34 describes a ventilator form factor compatible with a portable oxygen supply.

FIG. 35 describes a ventilator form factor compatible with a stationary compressor and oxygen concentrator.

FIG. 36 describes the overall system of the invention when the system is used to supply therapeutic gas to provide mechanical support to the patient.

FIG. 37 describes the overall system of the invention when the system is used to supply therapeutic gas to provide proper gas levels, and a pressurized air to provide mechanical breathing support.

FIG. 38 is a schematic view of the ventilator portion of the invention.

FIG. 39 describes a ventilator form factor compatible with mounting on a medical gas cylinder.

FIGS. 40-44 graphically describe the therapy as a function of time.

FIG. 40 describes a patient's lung pressure waveform.

FIG. 41 describes the ventilator flow delivery valve function.

FIG. 42 describes the gas flow delivery from the ventilator.

FIG. 43 describes the patient's lung volume as a result of the therapy.

FIG. 44 describes the operation of an optional breath detection sensor protection valve.

The various elements shown in FIGS. 1-44 are defined as follows: Pt: Patient or user; P: Proximal end of the ventilation cannula; D: Distal end of the ventilation cannula; d: Distance from gas delivery nozzle to nostril entrance; d′: Distance from superior side of interconnecting manifold to distal end of gas delivery nozzle; AS: Anterior side of the ventilation cannula; PS: Posterior side of the ventilation cannula; N: Nostril; V: Ventilator; G: Gas Supply; 2: Nostril septum; 9: Ventilation cannula with medial ventilation gas delivery port; 10: Ventilation Cannula with centered ventilation port and medial sensing port; 11: Ventilation Cannula with centered ventilation port and lateral sensing port; 12: Gas delivery nozzle of ventilation cannula; 14: Breath sensing tube of ventilation cannula; 20: Gas delivery tubing of ventilation cannula; 21: Breath sensing tubing of ventilation cannula; 22: Interconnecting manifold of ventilation cannula; 23: Manifold connectors; 24: Y-connector; 26: Nozzle opening of ventilation cannula; 28: Sensing tube opening of ventilation cannula; 30: Sensing tube chamfer; 31: CO2 sensing channel; 32: Ventilation gas flow channel; 33: Oxygen therapy delivery channel; 34: Breath sensing channel; 35: Breath sensing channel 2; 36: Ventilation Gas; 38: Entrained Gas; +: Positive Pressure; −: Negative Pressure; 40: Purge flow gas; 42: Oxygen therapy gas; 44: Expiratory Flow; 46: Nasal septum jet shield; 47: Venturi throat; 48: Dimple; 50: Sound dampening dimple; 52: Nozzle chamfer; 54: Gas flow tube diameter restriction; 56: Nozzle insulator; 58: Fluted nozzle tip; 60: Scalloped nozzle tip surface; 70: Volume Output Control Setting; 71: Delivery Time Control Setting; 72: Trigger Delay/Sensitivity; 73: Power Supply; 74: CPU; 75: Pressure Sensor, differential, flow control valve; 76: Pressure Sensor, breath effort detection; 77: Valve, optional breath effort detection (shuts off sensor to sensing line when volume is being delivered); 78: Flow Control Valve; 79: Regulator, electromechanical; 80: Medical gas source connection module; 81: Medical gas source connection knob/screw; 82: Gas delivery circuit to patient; 84: Ventilator; 85: Alarm panel; 86: Communication interface; 87: Gas compartment sealing wall; 88: Cylinder weight sensor module; 89: Weight sensor signal; 90: Medical gas cylinder; 91: Cylinder on/off valve; 92: Gas delivery circuit storage; 93: Weight sensor—Ventilator coupling; 94: Weight sensor wireless transmitter; 95: Weight sensor wireless receiver; 96: Weight sensor; 97: Outlet for gas delivery circuit connection; POC: Portable Oxygen Concentrator; LOX: Liquid Oxygen; PLOX: Portable Liquid Oxygen; -----: Electronic connection/signal; =. Pneumatic connection.

### DETAILED DESCRIPTION OF THE INVENTION

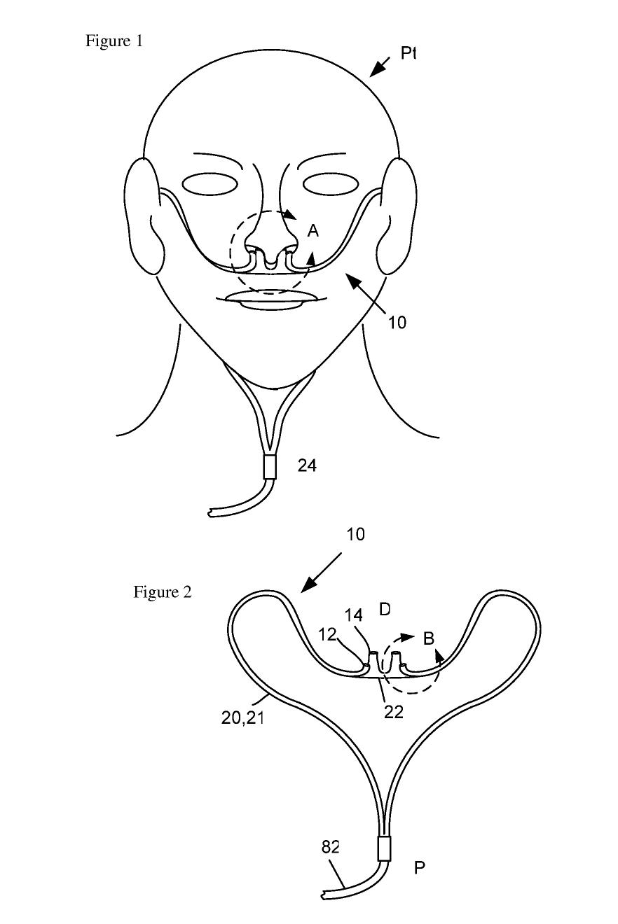

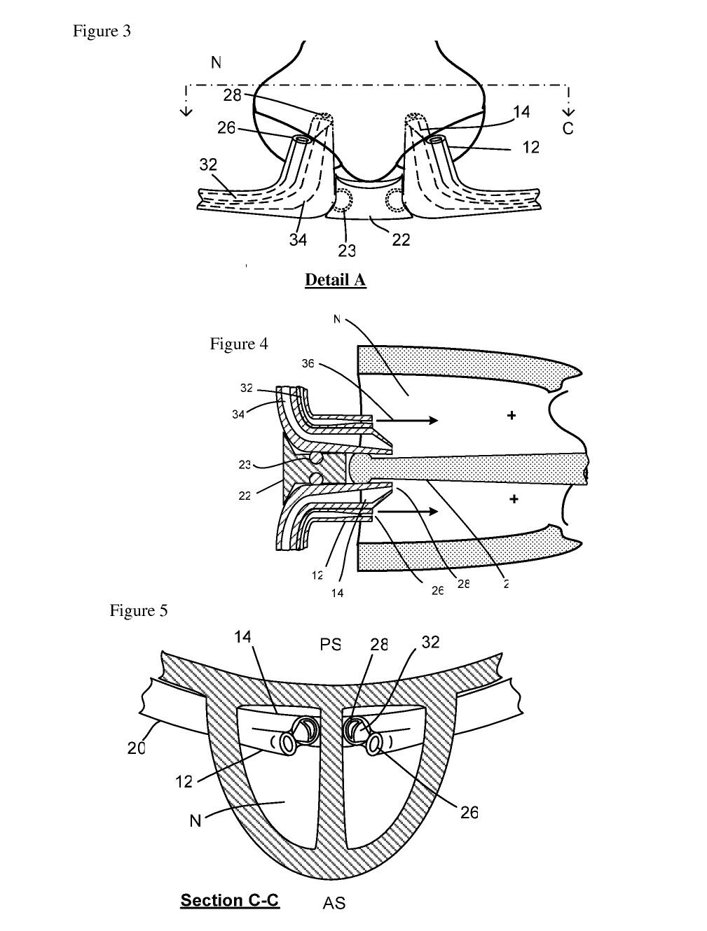

FIG. 1 describes the nasal ventilation cannula 10 being worn by a user Pt, and a Y connection 24. FIG. 2 shows an isometric view of the nasal ventilation cannula assembly 10 with gas delivery tubing 20 and breath sensing tubing 21 and interconnecting manifold 22, with a proximal end P with gas delivery circuit 82 which connects to a gas flow source and a distal end D which delivers gas flow to a patient's nasal airway via a ventilation gas delivery nozzle 12. User breathing is sensed via a sensing tube 14. FIG. 3 describes a detailed partial hidden live view of the distal end of the ventilation cannula 10 at detail A of FIG. 1. The gas delivery tube and breath sensing tubes are shown, as well as an interconnecting manifold 22 connected to the left and right sides. The interconnecting manifold can be adjustable or removable for example with connectors 23 in order to set the distance between the sensing tubes so that the sensing tubes hug the medial wall of the nostril N. The breath sensing tubes extend 1-10 mm into the nostrils, preferably 5-7 mm for adults, 3-5 mm for pediatrics, and 1-3 mm for neonates. The gas delivery nozzles 12 are coupled to the sensing tubes 14 such that the sensing tubes position the gas delivery nozzles laterally and anteriorly to the sensing tubes, at a position lateral to the medial wall of the nostril and offset from the skin. Typically, the gas delivery nozzles are located from 1-5 mm inside the entrance to the nostrils, although other positions are contemplated by the invention.

FIG. 4 shows a front view cross-section through the nose and ventilation cannula of the system shown in FIG. 3, showing the sensing tubes 14 pinching the nostril septum 2, the gas delivery nozzles 12 just lateral to the sensing tubes, and an interconnecting manifold 22 setting the distance between the sensing tubes. In the prior art described in US Patent Application No. 20110094518 the jet nozzles are positioned to the medial side of the sensing tubes, which positions the nozzles at an arbitrary location relative to the nostril anatomy, and in that particular case potentially too close to the nostril wall. An advantage of the present invention over the prior art is that the gas delivery nozzles are consistently positioned at an ideal location relative to the nostril's anatomical features, regardless of the patient's anatomy. Multiple sizes with different nozzle-to-nozzle spacings, or different sensing tube-to-sensing tube spacings, or with a spacing adjustment with a connector 23 are incorporated into the design to assure that the delivery nozzles are positioned at the ideal location. As can be seen in FIG. 4 the sensing tubes 14 position the ventilation gas flow 36 substantially in the center area of the nostril.

FIG. 5 describes a top view of the distal end of the nasal cannula in a user's nose, for example at the sectional view indicated by line C-C in FIG. 3. As can be seen, the sensing tubes 14 locate the tip of the gas delivery nozzles 12 lateral to the sensing tubes, and optionally slightly anteriorly. This position positively locates the gas delivery nozzle tips away from the wall of the nostrils N, and substantially centered in the nostril opening, so that the pressure profile exiting the gas flow nozzles 26 is centered or semi-centered with the nostril foramen. If the gas delivery nozzles are not centered, the gas flow unevenly impinges on a wall of the nostril which lessens the pressure generation and decreases the comfort of the gas flow. A chamfer 32 may be provided in the sensing tube 14 distal end to provide clearance to the gas exiting the nozzle openings 26. Optionally, the gas delivery nozzle openings 26 cross-sectional shape may be matched to match the relief or chamfer 32 in the sensing tube.

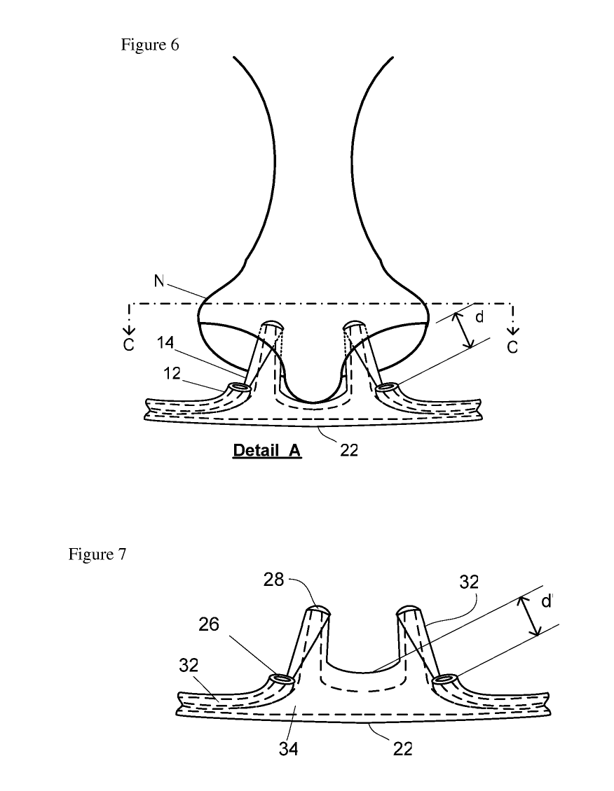

FIG. 6 describes a partial hidden line front view of an alternate ventilation cannula configuration in which the gas delivery nozzle 12 tips are located proximally to the entrance to the nostril, at a distance between 0 mm and 20 mm. Ideally, the distance d from the gas delivery nozzle to the entrance to the nostril is equal to one-half to three-fourth's the effective inside diameter of the nostril entrance during inspiration, or 8-10 mm for adults, 4-6 mm for pediatrics and 2-4 mm for infants. The interconnecting manifold 22 top surface is fit against the septum of the nostril to help position the sensing tubes and the gas delivery nozzles in their proper distance relative to the plane of the entrance to the nostrils to achieve the distances described above. The gas delivery nozzles tips are aligned to direct the gas exiting the nozzles along the centerline of the nostril foramen. The sensing tubes may be self-adjusting to the anatomy of the patient due to the compliance of the material, typically plastisol or silicone. The gas delivery nozzles resist deformation of their angular alignment with the nostril foramen, due to the semi-rigidity of their material, typically PVC or semi-rigid silicone. Alternately the ventilation cannula is provided in different sizes with an accompanying sizing guide so that the user can select a size that properly fits their nose.

FIG. 7 describes a closer view of the distal end of the ventilation cannula shown in FIG. 6. The nozzle opening 26 of the gas delivery nozzle is proximal or inferior to the superior or superior surface of the interconnecting manifold 22, therefore setting the dimensional relationship d′ between the gas delivery nozzle and the entrance to the nozzle. In order for the sensing tubes to not interfere with the gas flow exiting the gas delivery nozzle, the sensing tubes are chamfered 32 so that they are not in the gas delivery pathway, as will be described in more detail later. It is important that the sensing tubes hug the inside wall of the nostril, in order to assure that the gas delivery nozzle is properly positioned. Optionally, the interconnecting manifold can be removably attachable from the left and right cannula distal ends, so that the space between the left and right sensing tubes can be adjusted to fit the nose of an individual patient. Optionally, multiple sizes are made available for each patient group in order to meet the size requirements.

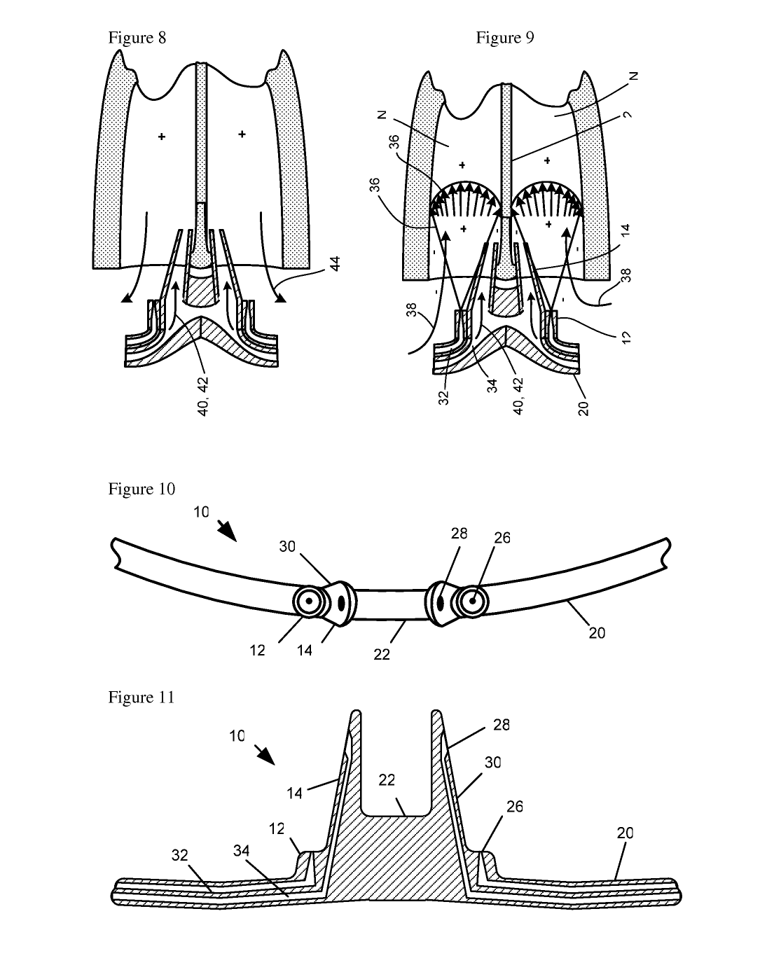

FIG. 8 describes a front sectional view through the nose of the distal end of the ventilation cannula, during expiratory phase of breathing. The patient's breath 44 is exhaling freely around the cannula. During expiratory phase, a purge flow of gas 40 can exit the sensing tubes to prevent occlusion of the tube with breathing fluids. The purge flow can be air or oxygen gas 42. Optionally the sensing tubes can be used to supply the oxygen gas required for maintaining the patient's oxygen saturation, and can be delivered continuously or intermittently. If continuous, the flow amplitude can increase during inspiratory phase and decrease during expiratory phase, or can be of constant amplitude. If intermittent, the gas can be delivered during inspiratory phase and switched off during expiratory phase. If PEEP is desired, gas can be delivered during expiratory phase. In FIG. 8 a diameter reduction in the gas delivery nozzle tip is shown to create the jet effect, and the sensing tube distal end is chamfered or angled to make clearance for the gas exiting the gas delivery nozzle.

FIG. 9 describes the view shown in FIG. 8 during inspiratory phase. A pressure head + is developed in the nostril at a distance inside the nostril. A negative pressure zone − is created outside of the pressure head, which entrains ambient air 38 into the nostril N to join with the gas delivered by the cannula, and the patient's spontaneously inspired ambient air (not depicted). A purge flow 40, 42 can be delivered through the breath sensing channel 34. The positive pressure zone of the gas delivery intersects with the wall of the nostril at a location inside the nose, typically 2 mm-10 mm inside from the opening, preferably 4-6 mm inside for adults, 3-4 mm for pediatrics, and 1-2 mm for infants. The jet gas delivery entrains ambient air as shown, typically 50-150% of the volume being supplied by the jets. For the adult sizes, the flow exiting the gas delivery nozzles is approximately 15 lpm for typically 0.75-1.0 second long bursts, and the entrained ambient airflow is 10-30 lpm depending on the prevailing conditions. Optionally, the gas being delivered by the gas delivery nozzles can be air to provide the mechanical breathing support, while oxygen gas is delivered through the sensing tubes for oxygenation.

FIG. 10 describes a top view of the distal end of a nasal ventilation cannula 10 with medial gas sensing tubes 14 and centered ventilation gas delivery nozzles 12 and FIG. 11 shows a front sectional view of the ventilation cannula shown in FIG. 10. Sensing tubes 14 placed medially pinch against the nostril septum and position the gas delivery nozzles 12 near the center of the nostril opening. In this variant, the sensing tube opening 28 is along the side of the tip of the sensing tube to help prevent it from being obscured and help it get cleaned by the ventilation gas blowing across it.

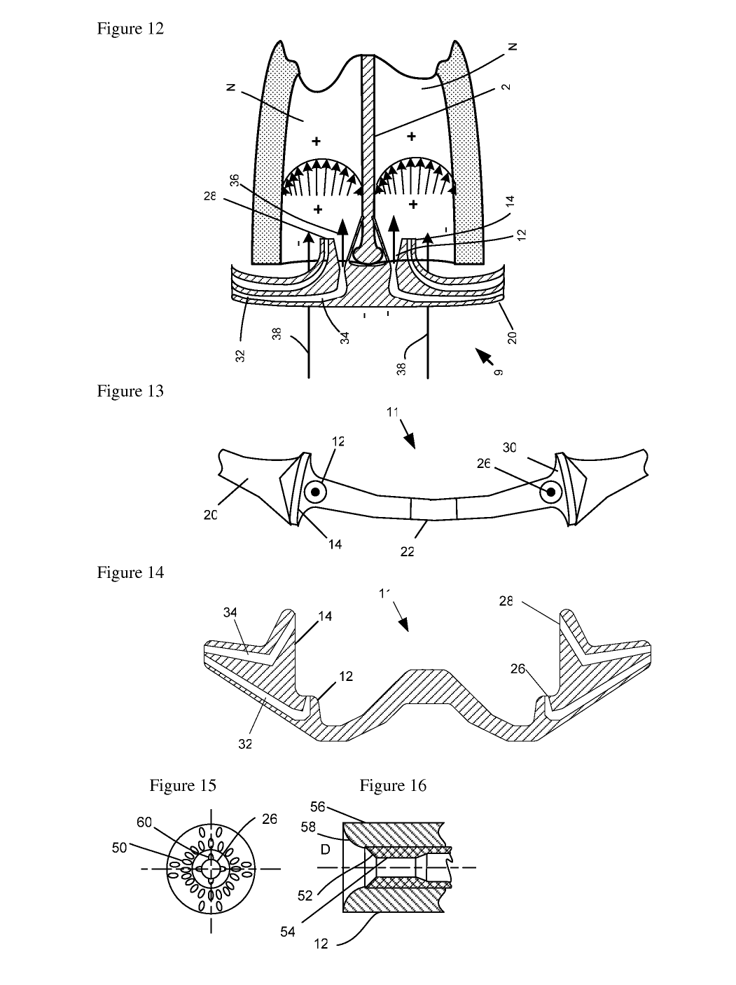

FIG. 12 shows a sectional front view of alternate ventilation cannula 9 to the cannula shown in FIG. 1 in which the gas delivery ports 12 are medial and the sensing ports 14 are centered. In this case the gas delivery tubes are configured to pinch against the nostril septum to properly position the nozzles along the medial wall of the nostril. An extension of the nozzle on the medial aspect of the nozzle protrudes into the nostril along the nostril wall to shield the sensitive tissue of the nostril in that area from the ventilation gas 36. The sensing tube opening are shown to be at the top aspect of the sensing tubes 14, however could also be placed on the side wall for example facing the ventilation gas delivery nozzle flow path.

FIG. 13 shows a top view of a ventilation cannula 11 with lateral sensing tubes 14 and centered ventilation gas delivery nozzles 12. FIG. 14 shows a front sectional view of the ventilation cannula shown in FIG. 13, showing the sensing ports 28 and gas delivery nozzle openings 26.

FIG. 16 describes a detailed sectional side view of the distal tip of the gas delivery jet nozzle 12. The gas delivery nozzle is shown with a restriction 54 for a length near the distal end, and a chamfer 52 at the distal tip. The restriction increases the velocity of the exiting gas, and the chamfer reduces the sound the gas generates when exiting the nozzle. The nozzle is encapsulated by an insulator 56, such as from material from the sensing tubes. The insulator can dampen the sound generated by the jet nozzle. The tip of the insulator is fluted 58 to help reduce eddy currents generated by the gas exiting the nozzle and therefore reduce sound and increase efficiency. FIG. 15 describes an end view of the distal tip of the gas delivery jet nozzle shown in FIG. 16. FIG. 15 describes an optional embodiment in which the inner surface of the tip of the nozzle and insulator are dimpled and scalloped. The dimples 50 and scallops 60 are employed to reduce the shear forces generated against the material and therefore reduce the sound that is generated and increase the efficiency in terms of pressure and flow creation at a given sound level. The material of the insulator can be comprised of a material that is especially known to dampen sound, for example comprising an absorptive substrate or a contact angle that cancels out the frequency and contact angle of the gas flow. The shape, orientation and distribution of the scallop features are determined by computational fluid mechanics and are proprietary. With these functional features, the resultant sound generated by the system can be in the range of 25-40 db and likely in the range of 30-35 db which will be within the range of acceptability and patient adherence.

The ventilation cannula is made of typically a thermoplastic or elastomeric compound, such as but not limited to PVC, plastisol, PCV-urethane blends, synthetic rubbers, silicone, urethane, or silicone-urethane blends. The jet nozzle subassembly is typically molded from a rigid thermoplastic such as Ultem or Delrin, or a semi-rigid thermoplastic such as PVC or polysolfone or semi-rigid silicone. The gas delivery channel and the nozzle can also be Teflon, boron, aluminum, and or magnesium impregnated to further reduce the coefficient of friction to reduce viscous drag at the boundary layers with gas flow. The gas delivery tubing is typically extruded using PVC or C-Flex or silicone. Dimensions of the ventilation cannula vary to make it compatible for neonatal, pediatric and adult patients, typically available in three sizes for each application. Additional straps can be added as necessary to secure the mask to the head and face as required.

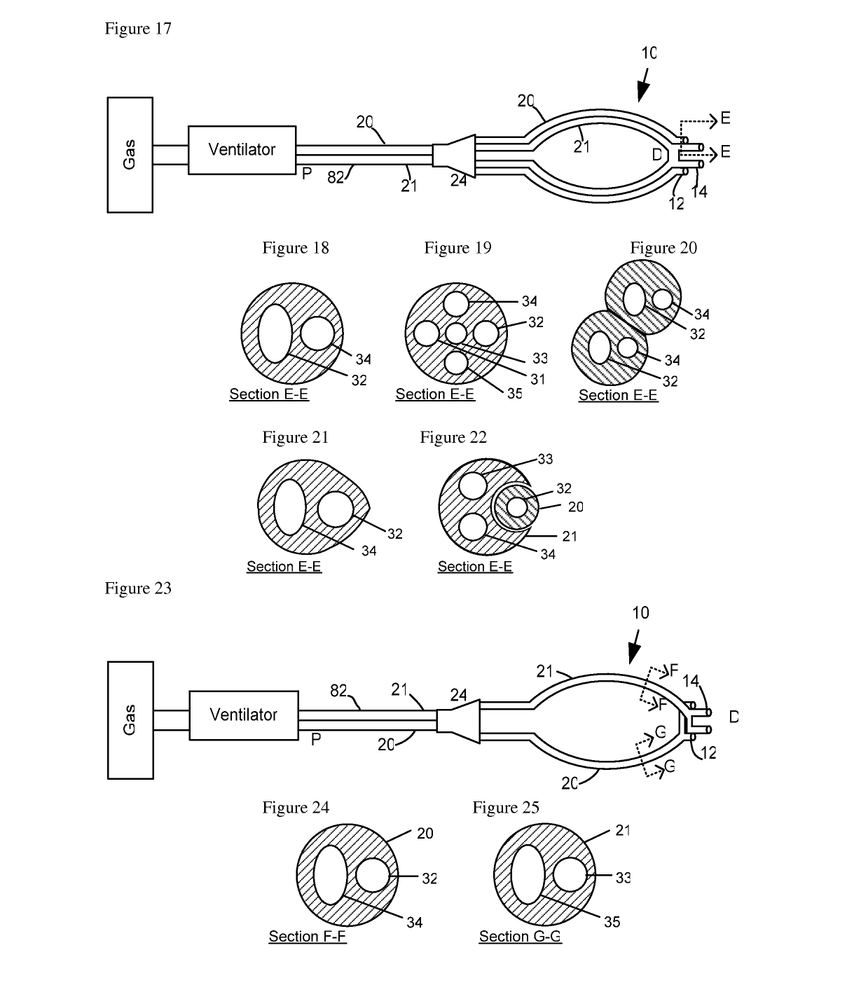

FIG. 17 describes a ventilation cannula 10 constructed from a dual lumen tube, with one lumen or channel assigned to the ventilation gas delivery, and a second lumen or channel assigned to the breath sensing tube and optionally purge flow, and optionally oxygen therapy as described earlier. FIGS. 18-22 show different cross sectional configurations of the invention, showing the breath sensing tube 21, the gas delivery tube 20, the CO2 sensing channel 31, the ventilation gas flow channel 32, the oxygen therapy delivery channel 33, and the breath sensing channel 34. FIG. 18 describes a cross-sectional view of the ventilation cannula at line E-E shown in FIG. 17, showing a dual lumen construction. FIG. 19 describes a cross-sectional view of the ventilation cannula at line E-E shown in FIG. 17, showing a quintuple lumen construction: The two breath sensing channels may terminate in sensing ports separated by a distance, so that flow direction and velocity can be determined. The gas composition channel can be used to monitor CO2 or FIO2 to help regulate the therapy. FIG. 20 describes an alternate cross-sectional view of the ventilation cannula showing a side-by-side tubing construction. FIG. 21 describes a cross-sectional view of the ventilation cannula at line E-E shown in FIG. 17, showing a non-round dual lumen construction. FIG. 22 describes an alternate cross-sectional view of the ventilation cannula showing a dual tube lumen construction at the distal tip, wherein the ventilation gas delivery tube is placed inside a pocket in the breath sensing tube. The sensing tube and gas delivery tube can be molded or formed separately, then assembled together.

FIG. 23 describes a ventilation cannula constructed from a dual tube configuration, with one tube assigned to ventilation gas delivery, and a second tube assigned to breath sensing, and optionally purge flow, and optionally oxygen therapy as described earlier. FIGS. 23 and 24 show alternative cross sectional configurations of FIG. 24 showing the breath sensing tube 21, the gas delivery tube 20, the ventilation gas flow channel 32, the oxygen therapy delivery channel 33, the breath sensing channel 34 and a second breath sensing channel 35. FIG. 24 describes a cross-sectional view of the dual tube construction ventilation cannula at line G-G shown in FIG. 23, showing the ventilation gas delivery tube cross-section. FIG. 25 describes a cross-sectional view of the dual tube construction ventilation cannula at line G-G shown in FIG. 23, showing the breath sensing tube cross-section.

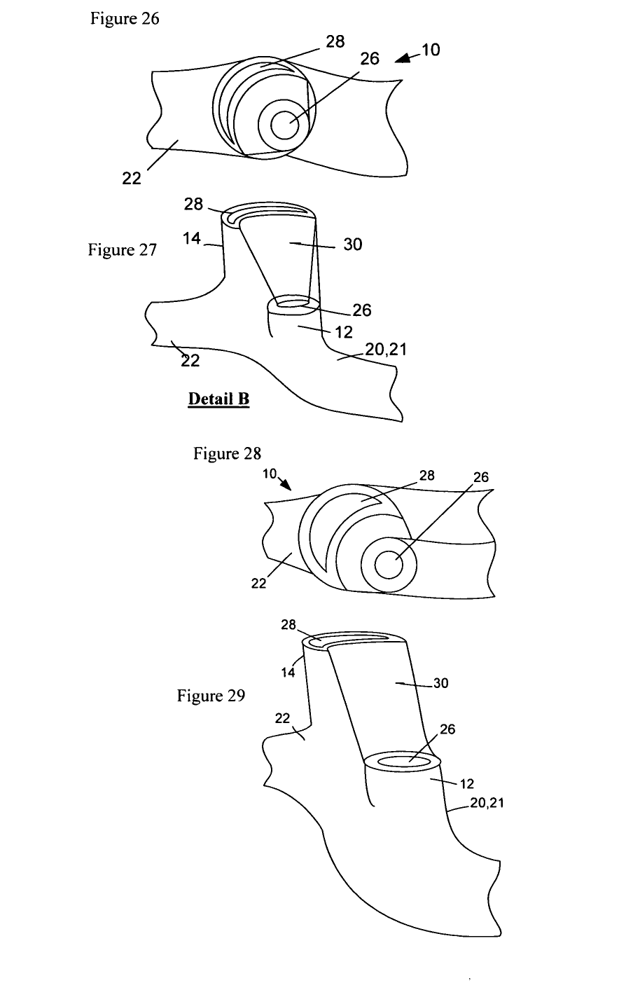

FIGS. 26-33 describe top and front views of one side of the distal tip of the ventilation cannula, showing the sensing tube and gas delivery nozzle. The gas delivery nozzle and sensing tube section of the ventilation cannula can be molded, then joined with the patient circuit tubing, or can be extruded and formed in a die, or a combination of molding, extruding, and die forming. FIG. 26 shows a top view of the left side of the distal end of the ventilation cannula 10 at Detail B shown in FIG. 2, showing a round dual lumen configuration with a lumen terminating with a breath sensing port 28 and a lumen terminating in the gas delivery nozzle 26. FIG. 27 shows a front view of the ventilation cannula detail shown in FIG. 26.

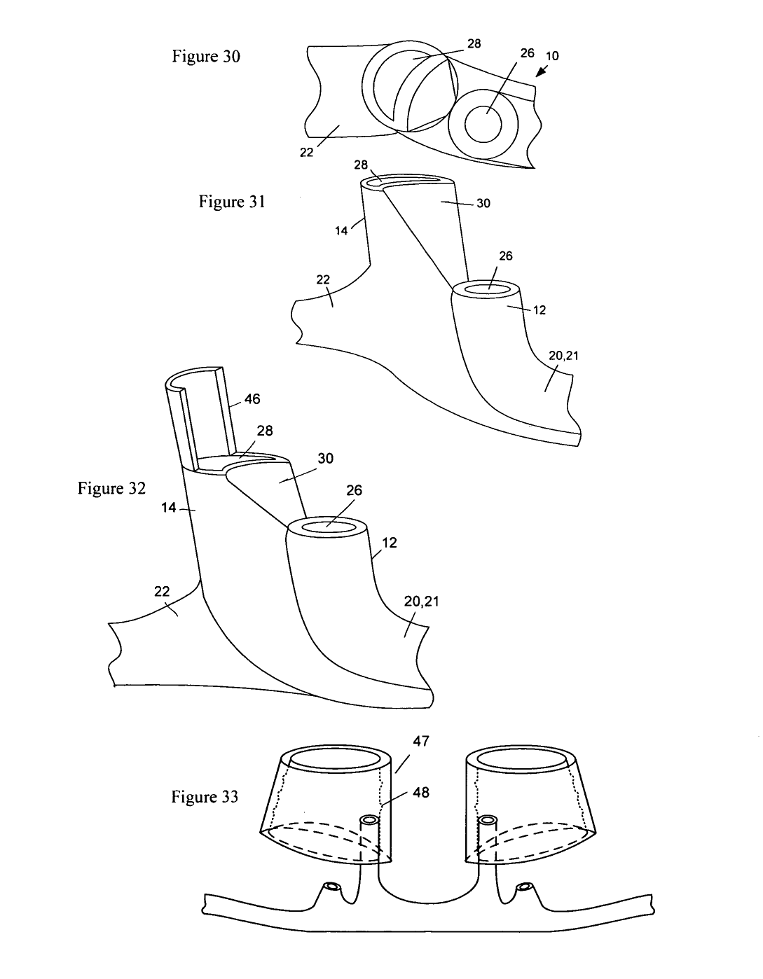

FIG. 28 shows a top view of the left side of the distal end of the ventilation cannula 10 at Detail B shown in FIG. 2, showing a non-round dual lumen configuration with a lumen terminating with a breath sensing port 28 and a lumen terminating in the gas delivery nozzle 26. FIG. 29 shows a front view of the ventilation cannula detail shown in FIG. 28. FIG. 30 shows a top view of the left side of the distal end of the ventilation cannula 10 at Detail B shown in FIG. 2, showing an adjoining dual tube configuration with a tube terminating with a breath sensing port 28 and a tube terminating in the gas delivery nozzle 26. FIG. 31 shows a front view of the ventilation cannula detail shown in FIG. 30. FIG. 32 describes a front view of the left side of the distal end of the ventilation cannula at Detail B shown in FIG. 2, showing a shield to shield the nostril inner wall from the jet gas flow.

FIG. 33 describes a front view of the distal end of a ventilation cannula with a Venturi throat coupled to the cannula. The Venturi throat can be connected to the sensing tube, and is dimensioned to fit into the nostril of the user. The throat is generally cylindrical, and can be radially expandable or compressible to permit a self fit to the interior dimensions of the nostril. The throat feature further enhances the performance of the Venturi jet pump of the ventilation cannula. The interior surface of the throat may be dimpled in order to reduce friction and shearing at the boundary layer between the gas flow and the throat wall, in order to reduce sound.

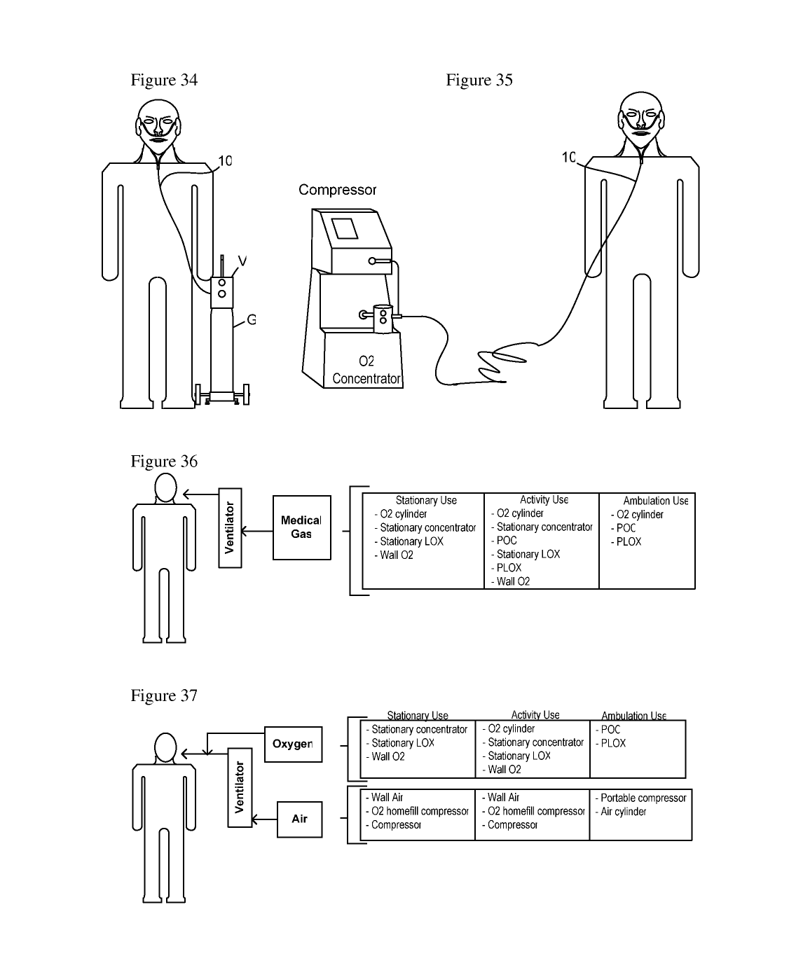

FIG. 34 describes a ventilator form factor compatible with a portable oxygen supply. In this configuration, the user can ambulate easily with the therapy. FIG. 35 describes a ventilator form factor compatible with a stationary compressor and oxygen concentrator. In this configuration, the user can use the therapy around their house with an extended length gas delivery circuit, for example while they are dressing. The mechanical support can be supplied by compressed air supplied by a compressor, and the oxygenation can be supplied by an oxygen concentrator. FIGS. 36 and 37 describe the different gas supply modalities that are used with the invention for stationary use, use during semi-stationary activity, and use during ambulation. In FIG. 36, oxygen is used to oxygenate and provide mechanical support. In FIG. 37, oxygen is used to provide oxygenation, and air is used to provide mechanical support. Optionally, a blender is used in conjunction with the ventilator to supply a desired FlO2 to the patient. The therapeutic gas can be oxygen, or other gas mixtures, such as heliox or NO mixtures.

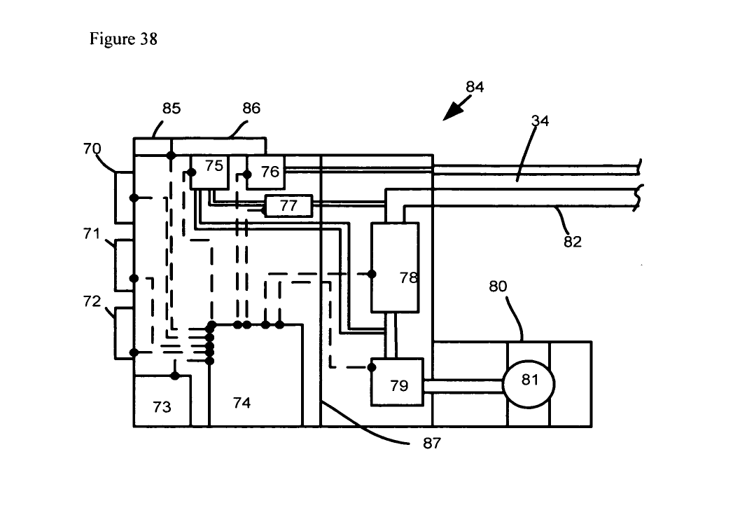

FIG. 38 is a schematic view of a ventilator used in conjunction with the nasal ventilation mask. Alternatively, the ventilator incorporates simple to use knob adjustments to adjust the volume output, the breath detection triggering sensitivity and the output pulse width. The ventilator typically has a rechargeable or replaceable internal battery, however can also work off of AC power. The ventilator can optionally be attachable to a medical gas source, such as an oxygen gas cylinder, using a medical gas connecting module. A sealed wall within the ventilator separates the medical gas handling componentry with the electronics and electricity used in the system, with only the necessary exceptions. The medical gas handling componentry may include an electro-mechanical gas pressure regulator and a flow control valve. A breath effort detection sensor is included to detect the onset of inspiration. The sensor measurement is relayed to the microprocessor, where control algorithms send the requisite signals to the flow control valve and control panel. The sensor is also used to detect an over pressure condition caused by a ventilator fault, or a situation at the patient. In the event the gas delivery lumen in the gas delivery tubing is also used for breath effort detection, and pressure sensor is coupled to the gas delivery lumen with a protection valve in between the sensor and the gas delivery conduit. Upon detection of the breath effort, the valve closes off the communication to protect the sensor from over-pressure detection. Typically the pressure output of the ventilator into the gas delivery circuit is 10-50 psi, and more typically 25-30 psi, in order to generate 5 cmH2O in the airways and lung, based on nominal conditions. In one embodiment of the invention, the pressure output is constant throughout the range of volume output settings, for example 25 psi. In this case the pulse width is changed as the volume setting is changed. In another embodiment, the pressure output is changed as the volume setting is changed. In yet another embodiment both the pressure output and pulse width are changed as the volume output setting is changed.

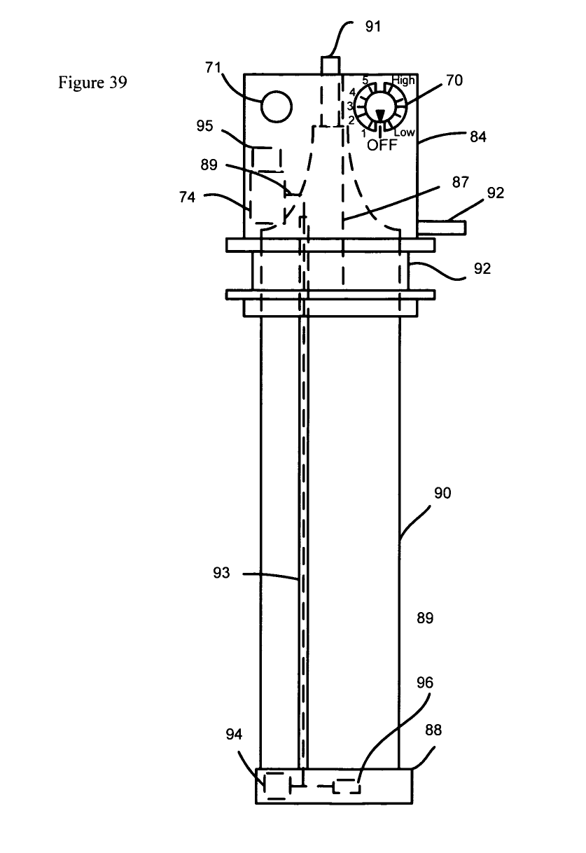

FIG. 39 describes an alternative embodiment of the invention in which the ventilator form factor is compatible with mounting on a medical gas cylinder, such as a compressed oxygen gas cylinder. This ventilator form factor may include a storage feature for the gas delivery circuit, or to store the excess length of the gas delivery circuit when the system is in use. A standard nasal cannula can also be stored in the storage feature. The storage feature can incorporate an automatic reeling feature for convenience to reel in the excess length and to reel out the needed length. In the example shown, the ventilator output selector is a knob type control, with settings from 1-5 lpm continuous flow output for when the patient requires standard oxygen therapy, and settings for multiple ventilator outputs volumes when the patient requires mechanical ventilatory support. The range of ventilator volume outputs will be prescribed by the prescribing physician and set into the ventilator using a physical or electronic key available to the attending clinical staff. Optionally, the ventilator includes a module for weighing the weight of the oxygen cylinder. The weight information is communicated with hard wire or wirelessly to the microprocessor in the ventilator. An algorithm uses this information, along with the pressure level in the cylinder, to compute the quantity of compressed oxygen remaining in the cylinder at any given time. The algorithm predicts the amount of time the oxygen cylinder can continue to be used by the patient, and informs the patient of this information.

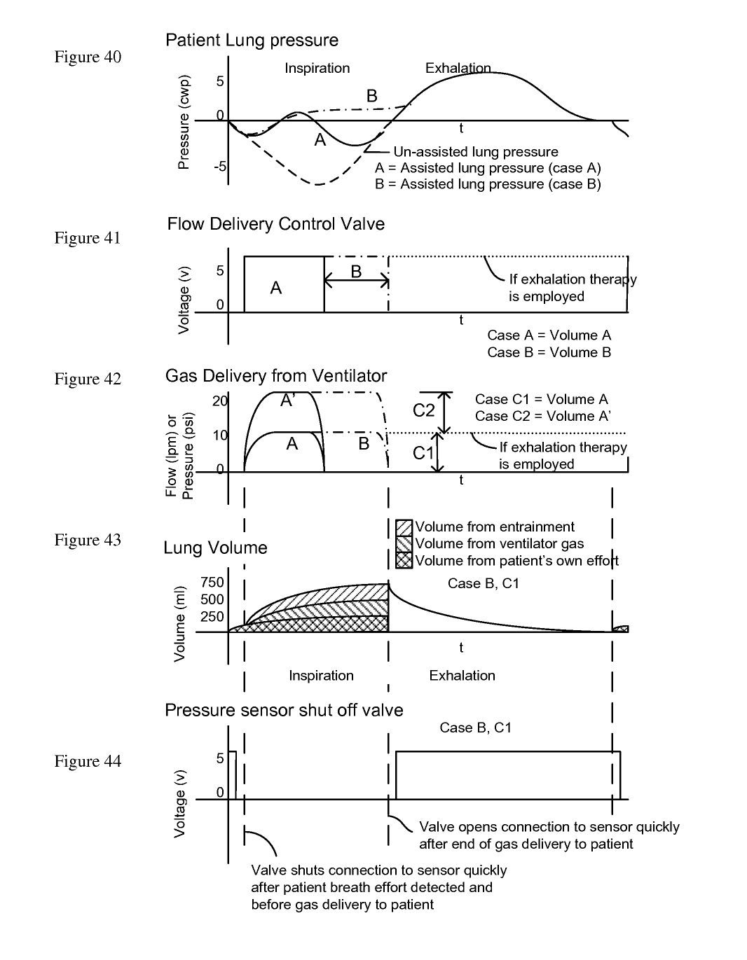

FIGS. 40-44 graphically describe the therapy as a function of time. FIG. 40 describes a patient's lung pressure waveform. For reference, the inspiratory waveform if the therapy was turned off is shown. Case A describes a pulse width in which the pulse time is a portion of the inspiratory time, for example 250 msec. Case B describes a longer pulse with intended to be roughly equal to the entire inspiratory time, and may therefore optionally be modulated to match the duration of the patient's inspiratory time. FIG. 41 describes the ventilator flow delivery valve function for Case A and Case B. The valve is normally closed to the patient outlet for safety purposes and power conservation. Upon an inspiratory effort created by the patient, the pressure sensor in the ventilator detects the effort, and signals the microprocessor to signal the flow control valve to open. The valve is then controlled based on the flow control algorithms in the microprocessor. The valve shown in the example has two states, open and closed, however the valve may be a variable orifice or variable position valve, controlled by a variable applied DC voltage or variable applied current, in order to control the output through the valve as desired. Pressure is measured on both sides of the valve to determine airflow and volume as well as pressure. Alternatively, when access to medical gas is not overly limited, gas flow can be delivered during exhalation phase to generate low levels of PEEP to assist in exhalation. FIG. 42 describes the gas flow delivery from the ventilator for Cases A and B, and Cases C1 and C2. C1 is a setting for a volume setting of for example 100 ml, and C2 is a setting for a volume setting of for example 200 ml. FIG. 43 describes the patient's lung volume as a result of the therapy indicating the constituent sources of the gas. FIG. 44 describes the pressure sensor protection valve function in the case that the gas delivery channel is also used for breath effort detection.

The volume output of the ventilator is typically 25-500 ml per cycle for adults, more typically 50-175 ml. The exit speed of the gas exiting the nozzle is typically 50-400 m/sec, more typically 100-250 m/sec. The ambient air entrained by the Venturi is typically 25-200%, more typically 50-100%. The pressure generated by the system in the upper airway can be 1-20 cmH2O and in the lung 1-15 cmH2O above non-assisted pressures, and typically in the range of 5-12 cmH2O and 3-8 cmH2O respectively. The dimensions of the gas delivery nozzle are 0.010″ to 0.030″ in effective internal diameter, and the breath sensing port is 0.015-0.040 mm in effective internal diameter. The overall cross-sectional dimension of the ventilation cannula tip for adult sizes, including the sensing tube and gas delivery tube, is approximately 0.175-250″ in effective outside diameter, compared to 0.210″ outer diameter that is typical of a standard adult oxygen nasal cannula, therefore resulting in a fully functional ventilation interface that is approximately the same size of a standard oxygen nasal cannula.

Additional aspects of the invention include the following. A ventilation apparatus comprising a nasal ventilation cannula, and gas delivery system, wherein the ventilation cannula comprises: a proximal end adapted to attach to the gas delivery system and a distal end adapted to engage with the nares, a sensing tube comprising a distal end configured to enter a nostril, a gas delivery channel comprising a distal end coupled to the lateral aspect of sensing tube and terminating with a gas delivery nozzle at the distal end. A ventilation apparatus comprising a nasal ventilation cannula, and gas delivery system, wherein the ventilation cannula comprises: a proximal end adapted to attach to the gas delivery system and a distal end adapted to engage with the nares; a sensing tube comprising a distal end configured to enter a nostril, a gas delivery channel comprising a distal end coupled to the lateral aspect of sensing tube and terminating with a gas delivery nozzle at the distal end; and wherein the gas exiting the gas delivery nozzle creates an expanding gas flow profile entering the nostrils, and wherein the distal end of the sensing tube comprises an indentation along the outside of the tube which is configured to allow clearance for the gas flow profile. A ventilation cannula wherein the distal end of the gas delivery tube is further coupled to the anterior aspect of the sensing tube. A ventilation cannula wherein the gas delivery nozzle is coupled to the sensing tube at a distance proximally from the distal tip of the sensing tube, resulting in a nozzle position below and outside of the nostril. A ventilation cannula wherein gas exiting the gas delivery nozzle entrains ambient air into the nasal airway. A ventilation cannula wherein oxygen enriched gas is delivered into the nasal airway through the gas delivery nozzle, to treat for example COPD or ILD. A ventilation cannula wherein oxygen gas is delivered through the sensing tube at a low pressure low velocity level to maintain oxygen saturation and air is delivered at high pressure and high velocity through the gas delivery nozzle to provide mechanical support to the lung. A ventilation cannula wherein oxygen gas is delivered through the sensing tube and through the gas delivery nozzle. A ventilation cannula wherein air is delivered through the gas delivery nozzle to provide mechanical support. A ventilation system further comprising a ventilator configured to adapt to an oxygen gas cylinder. A ventilation cannula further comprising a ventilator configured to adapt to an oxygen concentrator. A ventilation cannula further comprising a ventilator configured to adapt to a gas compressor. A ventilation cannula wherein the distal tip of the gas delivery nozzle further comprising depressions configured to dampen sound. A ventilation cannula wherein the distal tip of the gas delivery nozzle further comprising a scalloped inner diameter at the end configured to reduce shearing. A ventilation cannula wherein the distal tip of the gas delivery nozzle tip is recessed inside the nostril entrance, from 0.1-5.0 mm recessed. A ventilation cannula wherein the distal tip of the gas delivery nozzle tip is co-planar with the nostril entrance. A ventilation cannula wherein the distal tip of the gas delivery nozzle tip is proximal to the nostril entrance. A ventilation cannula wherein the distal tip of the gas delivery nozzle tip is between 0.25″ and 0.75″ proximal to the entrance to the nostril. A ventilation cannula wherein the distal tip of the gas delivery nozzle tip is a distance from the entrance to the nostril equal to about one-third to three-fourths of the nostril entrance effective diameter, and wherein the tip of the gas delivery nozzle inner diameter is flared wider to emit a flow path such that the conical flow path intersects with the nostril inner wall at a distance inside the nostril from 1 mm to 10 mm from the nostril entrance. A ventilation cannula wherein the gas delivery nozzle cross-section is non-round to match the cross-sectional anatomy of the nostril. A ventilation cannula wherein the ventilation cannula is constructed from a dual lumen tube, with one lumen as the sensing tube, and one lumen as the gas delivery lumen. A ventilation cannula wherein the ventilation cannula is constructed from two tubes, with one tube as the sensing tube, and another tube as a gas delivery tube. A ventilation cannula wherein the ventilation cannula distal tip further comprises a shield adapted to be placed against a portion of the inside of the nostril wall. A ventilation cannula wherein the ventilation cannula comprises a flow of gas in the sensing channel to maintain a patent channel. A ventilation cannula further comprising (1) a second breath sensing port positioned proximally to the first breath sensing port, and (2) a gas composition sensing port and channel. A ventilation cannula further comprising a Venturi pump throat section, the section comprising a substantially cylindrical tube coupled to the ventilation cannula distal end and adapted to be inserted into the nostril of the user.

Additional aspects of the invention also include the following. A method for providing respiratory support at a low cost that is negligibly incremental to current spending in order to allow widespread use, the method comprising: adapting a standard nasal oxygen therapy cannula into a ventilation cannula by adding to the cannula a ventilation gas delivery channel and nozzle, using the oxygen delivery prongs of the nasal cannula as breath sensing prongs, positioning the added gas delivery nozzle near the entrance to the nostrils by coupling it proximal to the tips of the cannula prongs, and delivering gas through the nozzles at a velocity to create a positive pressure of greater than 5 cmH2O inside the nasal airway. A method further wherein the system is used for a stationary treatment session in the hospital setting to rest the respiratory muscles to make the patient more tolerant to exertion after a treatment session, wherein the system is connected to a wall oxygen supply. A method further wherein the system is used in the hospital setting during semi-stationary activity, such as moving around the hospital room, or participating in a physical or occupational therapy session at the bedside or in a therapy room, wherein the system is connected to a hospital wall oxygen supply. A method further wherein the system is used in the hospital setting to enable ambulatory use, such as enabling the patient to walk to another department within the hospital, wherein the system is connected to a compressed oxygen cylinder. A method further wherein the system is used during an exercise session in the institutional setting to condition the respiratory muscles to improve the patient's pulmonary mechanics, wherein the system is connected to a compressed oxygen cylinder. A method further wherein the system is used for a treatment session in the emergency setting to alleviate dyspnea and provide a level of ventilatory support, wherein the system is connected to a compressed oxygen supply. A method further wherein the system is used for a stationary treatment session in the home setting to rest the respiratory muscles to make the patient more tolerant to exertion after a treatment session. A method further wherein the system is used in the home setting during semi-stationary activity, such as bathing, wherein the system is connected to a stationary oxygen concentrator or compressor system with an extended tubing length. A method further wherein the system is used in the home or community setting to enable ambulatory use, wherein the system is connected to a compressed oxygen cylinder or portable oxygen supply. A method further wherein the system is used during an exercise session in the home or community setting to condition the respiratory muscles to improve the patient's pulmonary mechanics, wherein the system is connected to an oxygen concentrator or compressor or compressed oxygen supply. A method wherein compressed air is supplied through the gas delivery nozzle for mechanical support and oxygen is supplied for oxygenation. A method wherein the system is connected to a blended oxygen-air mixture is supplied to regulate blood gas levels.

As part of the present invention, it should be noted that the embodiments and elements described in the specification can be applied to the invention in part and in any reasonable combination, and for brevity not all such permutations and combinations are explicitly described.

What is claimed is:

1. A ventilation apparatus comprising a nasal ventilation cannula, and gas delivery system, wherein the ventilation cannula comprises:
a. A proximal end adapted to attach to the gas delivery system and a distal end adapted to engage with the nares,
b. A sensing tube comprising a distal end configured to enter a nostril, a medial aspect directed toward the nostril septum, a lateral aspect directed toward the nostril lateral wall, an anterior aspect directed away from the skin, and a posterior aspect directed toward the skin,
c. A gas delivery channel comprising a distal end coupled to the lateral aspect of sensing tube and terminating with a gas delivery nozzle at the distal end.

2. A ventilation apparatus comprising a nasal ventilation cannula, and gas delivery system, wherein the ventilation cannula comprises:
a. A proximal end adapted to attach to the gas delivery system and a distal end adapted to engage with the nares;
b. A sensing tube comprising a distal end configured to enter a nostril, the distal end comprising a medial aspect directed toward the nostril septum, a lateral aspect directed toward the nostril lateral wall, an anterior aspect directed away from the skin, and a posterior aspect directed toward the skin,
c. A gas delivery channel comprising a distal end coupled to the lateral aspect of sensing tube and terminating with a gas delivery nozzle at the distal end;
and wherein the gas exiting the gas delivery nozzle creates an expanding gas flow profile entering the nostrils, and wherein the distal end of the sensing tube comprises an indentation along the outside of the tube which is configured to allow clearance for the gas flow profile.

3. A ventilation cannula as described in claim 2 wherein the distal end of the gas delivery tube is further coupled to the anterior aspect of the sensing tube.

4. A ventilation cannula as described in claim 2 wherein the gas delivery nozzle is coupled to the sensing tube at a distance proximally from the distal tip of the sensing tube, resulting in a nozzle position below and outside of the nostril.

5. A ventilation cannula as described in claim 2 wherein gas exiting the gas delivery nozzle entrains ambient air into the nasal airway.

6. A ventilation cannula as described in claim 2 wherein oxygen enriched gas is delivered into the nasal airway through the gas delivery nozzle.

7. A ventilation cannula as described in claim 2 wherein oxygen gas is delivered through the sensing tube at a low pressure low velocity level to maintain oxygen saturation and air is delivered at high pressure and high velocity through the gas delivery nozzle to provide mechanical support to the lung.

8. A ventilation cannula as described in claim 2 wherein oxygen gas is delivered through the sensing tube and through the gas delivery nozzle.

9. A ventilation cannula as described in claim 2 wherein air is delivered through the gas delivery nozzle to provide mechanical support.

10. A ventilation system as described in claim 2 further comprising a ventilator configured to adapt to an oxygen gas cylinder.

11. A ventilation cannula as described in claim 2 further comprising a ventilator configured to adapt to an oxygen concentrator.

12. A ventilation cannula as described in claim 2 further comprising a ventilator configured to adapt to a gas compressor.

13. A ventilation cannula as described in claim 2 wherein the distal tip of the gas delivery nozzle tip is recessed inside the nostril entrance, from 0.1-5.0 mm recessed.

14. A ventilation cannula as described in claim 2 wherein the distal tip of the gas delivery nozzle tip is co-planar with the nostril entrance.

15. A ventilation cannula as described in claim 2 wherein the distal tip of the gas delivery nozzle tip is proximal to the nostril entrance.

16. A ventilation cannula as described in claims 1 and 2 wherein the distal tip of the gas delivery nozzle tip is between 0.25″ and 0.75″ proximal to the entrance to the nostril.

17. A ventilation cannula as described in claim 2 wherein the distal tip of the gas delivery nozzle tip is a distance from the entrance to the nostril equal to about one-third to three-fourths of the nostril entrance effective diameter, and wherein the tip of the gas delivery nozzle inner diameter is flared wider to emit a flow path such that the conical flow path intersects with the nostril inner wall at a distance inside the nostril from 1 mm to 10 mm from the nostril entrance.

18. A ventilation cannula as described in claim 2 wherein the gas delivery nozzle cross-section is non-round to match the cross-sectional anatomy of the nostril.

19. A ventilation cannula as described in claim 2 wherein the ventilation cannula is constructed from a dual lumen tube, with one lumen as the sensing tube, and one lumen as the gas delivery lumen.

20. A ventilation cannula as described in claim 2 wherein the ventilation cannula is constructed from two tubes, with one tube as the sensing tube, and another tube as a gas delivery tube.
